(19) United States
(12) Patent Application Publication
(10) Pub. No.: US 2004/0248982 A1
(43) Pub. Date: Dec. 9, 2004

(54) Use of compounds for the inhibition of proteins and UV protection

(76) Inventor: Gordon Wayne Dyer, Chesterfield, MO (US)

Correspondence Address: Gordon Wayne Dyer, 19269 Babler Forest Rd., Chesterfield, MO, 63005, US

(21) Appl. No.: 10/458,155

(22) Filed: Jun. 9, 2003

(51) Int. Cl. A61K 31/195; A61K 31/19

(52) U.S. Cl. 514/566; 514/574; 514/315; 514/355; 514/310; 514/406; 514/311

(57) ABSTRACT

The invention treats proteinase-related disorders by interfering with the binding of the metal needed by matrix metalloproteinases, such as PLA-2. The compounds used are known microbial-based chelators of known high solubility. For example, dipicolinic acid has a high affinity to calcium and is soluble in both water and glycerin, as might be expected due to its role in the formation of bacterial endospores. Thus, unlike most chelation agents, the compounds are intended to be permeable enough to cross a vertebrate's cell membrane, a prerequisite for meaningful inhibition of protein-regulated cellular functions such as the inflammatory cascade and accompanying formation of free fatty acids. In addition, because six-ring aromatic chemical structures are inherently durable, it is likely that these compounds will be long-acting, an important attribute because most proteinase-related disorder/diseases are chronic

### BACKGROUND OF THE INVENTION

1. Field of the Invention

The present invention discloses compounds that increase ultraviolet radiation protection and decrease the action of some proteins. More particularly, the present invention provides anti-proteinase compounds designed to be capable of crossing a cell membrane.

2. Description of Related Art

Many chronic and acute protein-mediated diseases and disorders are characterized by increased intracellular levels of free metals. These metals are directly damaging to the cell by increasing intracellular free radical formation which damage cell content due to the fact that they are a reactive oxygen species, (ROS), the Fenton reaction. These metals also indirectly cause damage both extracellularly and to the cell itself due to, for example, their role in the inflammatory cascade when coupled to the appropriate cellular protein as part of a matrix metalloproteinase (MMPs). These proteins are somewhat unique in that the protein used in the formation of an MMP is of a transient nature. Without the addition of the free metal the protein is recycled by the cell into other cellular products. Such protein-mediated disorders include rheumatoid arthritis, osteoarthritis, atherosclerosis, pulmonary emphysema, acne, Lupus, Alzheimer's disease, sterile corneal ulcer, iritis, blepharitis, aneurysm formation, tumor metastasis, and tumor angiogenesis.

Classic metal chelators, such as EDTA, have been touted, particularly in Europe, as medically therapeutic agents but their utility is highly questionable due to the fact that the very character that makes them such excellent long lasting chelators, their strong ionicity, makes them too lipid insoluble to be capable of crossing the cell membrane necessary to inhibit any MMP activity. In addition, strong chelators work have a preference for heavy metals, but the metals used in MMPs range from light metals to transition metals.

Recently, it was discovered that tetracyclines, particularly chemically modified tetracyclines (CMT), are, among other useful properties, effective anti-proteinase agents due to their ability to bind free metals, particularly calcium. CMTs are tetracyclines that have been shorn or the chemical subgroup that gives tradition tetracyclines their anti-microbial property. Ironically, removal of this anti-microbial subgroup apparently enhances the CMTs ability to bind metal, yet the CMTs retain their aromatic ring-based ability to cross cell membranes.

Unfortunately, all tetracyclines, particularly CMTs become free radicals upon exposure to light. These tetracycline-derived free radicals, particularly CMTs, then proceed to cause cellular damage via the Fenton reaction already mentioned above predisposing the patient to severe sunburn. In addition, use of CMTs to help control protein related diseases/disorders has two inherent problems because of the chronic nature of these disorders. First, continued use of tetracyclines as anti-microbial will, due to their chemical similarities to CMTs lead to an increasing number of endemic bodily microbes that, to protect themselves, produce compounds that bind all tetracycline-based chemicals, thus impairing the CMTs future anti-proteinase effect. Secondly, tetracyclines are unstable, easily metabolized chemicals.

It would therefore be desirable to provide compounds that, like CMTs, preferentially chelate light-to-transition metals, have no chemical similarity to any anti-microbial agents and yet can penetrate cell membranes to reduce MMP activity by binding and/or interfering with free metals, but that would have a greater affinity for calcium, increase rather than decrease sunburn resistance, and would be longer lasting due to having inherently stronger chemical structures than do tetracyclines.

### SUMMARY

In accordance with the present invention, compounds are provided for the treatment of diseases and disorders that are mediated by proteins, including but not limited to, rheumatoid arthritis, osteoarthritis, atherosclerosis, pulmonary emphysema, acne vulgaris, acne rosacea, lupus erythematosis, Alzheimer's disease, sterile corneal ulcer, iritis, blepharitis, aneurysm formation, tumor metastasis, over production of C-reactive protein, and tumor angiogenesis that require metals to be bound to them before they are functional. The present invention includes preventing the metals from effectively joining with the proteins, thereby inhibiting the function of the protein for the treatment of protein-mediated vertebrate diseases and disorders.

The present invention also includes the use of ultraviolet (UV) absorbing compounds, capable of crossing a vertebral cell membrane, to inhibit a vertebrate from sunburning. Because of their ability to cross a cell membrane, the present invention contemplates the use of pill, as well as capsule liquid, aerosol, powder, spray, mist, creme, ointment, atomized vapor, or suspension, to inhibit, in a safe and effective manner, a vertebrate from sunburning.

The present invention includes treating protein-mediated vertebrate diseases and disorders, using chemical compounds capable of electronegatively interacting with a metallic cation in a manner and amount sufficient to substantially impair the metallic cation from effectively joining with the protein, thereby inhibiting the function of the protein for the treatment of protein-mediated vertebrate diseases and disorders.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to electronegatively interact with a metallic cation in a manner and amount sufficient to substantially impair the metallic cation from effectively joining with the protein, thereby inhibiting the function of the protein, in a safe and effective manner, for the treatment of protein-mediated vertebrate diseases and disorders.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to electronegatively interact with a metallic cation in a manner and amount sufficient to substantially impair the metallic cations from effectively joining with a protein for the treatment of protein-mediated vertebrate diseases and disorders, wherein the carboxylic or dicarboxylic acid that chelates the metal from joining with the protein is mixed with a base, the acid ranging from 0.01% to 99.99% of the mixture and the base ranging from 0.01% to 99.99% of the mixture.

The present invention includes using compounds that are, in whole or in part, 5 or 6 member cyclical ring carboxylic or dicarboxylic acids, having at least one of the ring member that is of an electronegatively inductive nature or has an electronegatively inductive carbonyl subgroup, to electronegatively interact with a metallic cation in a manner and amount sufficient to substantially impair the metallic cation from effectively joining with the protein, thereby inhibiting the function of the protein, for the treatment of protein-mediated vertebrate diseases and disorders, wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to electronegatively interact with a metallic cation in a manner and amount sufficient to substantially impair the metallic cation from effectively joining with the protein, thereby inhibiting the function of the protein for the treatment of protein-mediated vertebrate diseases and disorders, wherein the protein in question is a nascent or existing matrix metalloprotein and the pH of the mixture is selected to optimize the ability of the mixture to cross the cell membrane of the vertebrate, in a safe and effective manner, and wherein the base is selected from the group consisting of ammonia cation, amine cation, and hydrazine cation.

The present invention also includes having the pH of the mixture used to electronegatively interact with a metallic cation in a manner and amount sufficient to substantially impair, in a safe and effective manner, the metallic cation from effectively joining with the protein, thereby inhibiting the function of the protein for the treatment of protein-mediated vertebrate diseases and disorders be selected to optimize the ability of the mixture to cross the cell membrane of the vertebrate and having the base be selected from the group consisting of ammonia cation, amine cation, and hydrazine cation.

The present invention includes administering chemical compounds for treating protein-mediated vertebrate diseases and disorders by chelating metallic cations in a manner and amount sufficient to substantially impair, in a safe and effective manner, the metallic cation from effectively joining with protein.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to chelate metallic cations in a manner and amount sufficient to substantially impair, in a safe and effective manner, the metallic cation from effectively joining with a protein for the treatment of protein-mediated vertebrate diseases and disorders.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to chelate metallic cations in a manner and amount sufficient to substantially impair, in a safe and effective manner, the metallic cations from effectively joining with a protein for the treatment of protein-mediated vertebrate diseases and disorders, wherein the carboxylic or dicarboxylic acid that chelates the metal from joining with the protein is mixed with a base, the acid ranging from 0.01% to 99.99% of the mixture and the base ranging from 0.01% to 99.99% of the mixture.

The present invention also includes using compounds that are, in whole or in part, or 6 member cyclical ring carboxylic or dicarboxylic acids, having at least one of the ring member that is of an electronegatively inductive nature or has an electronegative carbonyl subgroup, to chelate a metallic cations in a manner and amount sufficient to substantially impair the metallic cation from effectively joining with a protein for the treatment of protein-mediated vertebrate diseases and disorders in a safe and effective manner, wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to chelate metallic cations in a manner and amount sufficient to substantially impair the metallic cation from effectively joining with a protein for the treatment of protein-mediated vertebrate diseases and disorders in a safe and effective manner, wherein the protein in question is a nascent or existing matrix metalloprotein and the pH of the mixture is selected to optimize the ability of the mixture to cross the cell membrane of the vertebrate, in a safe and effective manner, and wherein the base is selected from the group consisting of ammonia cation, amine cation, and hydrazine cation.

The present invention includes treating protein-mediated vertebrate diseases and disorders by administering a chemical compound capable of sequestering a metallic cation within a complex in a manner and amount sufficient to substantially impair, in a safe and effective manner, the metallic cation from effectively joining with the protein, thereby inhibiting the function of the protein.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to sequester metallic cations within a complex in a manner and amount sufficient to substantially impair the metallic cation from effectively joining with a protein for the treatment of protein-mediated vertebrate diseases and disorders.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to sequester metallic cations within a complex in a manner and amount sufficient to substantially impair the metallic cation from effectively joining with a protein for the treatment of protein-mediated vertebrate diseases and disorders, wherein the carboxylic or dicarboxylic acid that sequesters the metal from proximity with the protein is mixed with a base, the acid ranging from 0.01% to 99.99% of the mixture and the base ranging from 0.01% to 99.99% of the mixture.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to sequester a metallic cations within a complex in a manner and amount sufficient to substantially impair the metallic cation from effectively joining with a protein for the treatment of protein-mediated vertebrate diseases and disorders, wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

The present invention also includes using compounds that are, in whole or in part, 5 or 6 member cyclical ring carboxylic or dicarboxylic acids, having at least one of the ring member that is of an electronegatively inductive nature or has an electronegative carbonyl subgroup, to sequester metallic cations within a complex in a manner and amount sufficient to substantially impair the metallic cation from effectively joining with a protein for the treatment of protein-mediated vertebrate diseases and disorders, wherein the protein in question is a nascent or existing matrix metalloprotein and the pH of the mixture is selected to optimize the ability of the mixture to cross the cell membrane of the vertebrate and wherein the base is selected from the group consisting of ammonia cation, amine cation, and hydrazine cation.

The present invention also includes a method for treating protein-mediated vertebrate diseases and disorders, by administering a chemical compound capable of interacting with a metallic cation in a manner and amount sufficient to substantially impair the metallic cation from effectively joining with the protein., wherein the compound is formulated for administration from the group consisting of pill, capsule, liquid, aerosol, powder, spray, mist, creme, ointment, atomized vapor, and suspension.

The present invention includes treating protein-mediated vertebrate diseases and disorders by administering, in whole or in part, a 5 or 6 member cyclical ring carboxylic or dicarboxylic acid chelator which contains at least one ring members that is of an electronegatively inductive nature, wherein the compound is formulated for administration from the group consisting of pill, capsule, liquid, aerosol, powder, spray, mist, creme, ointment, atomized vapor, and suspension.

The present invention also includes treating protein-mediated vertebrate diseases and disorders by administering a chemical compound capable of sequestering a metallic cation within a complex in a manner and amount sufficient to substantially impair the metallic cation from effectively joining with the protein, wherein the compound is formulated for administration from the group consisting of pill, capsule, liquid, aerosol, powder, spray, mist, creme, ointment, atomized vapor, and suspension.

The present invention also includes compounds for the treatment of protein-mediated vertebrate diseases and disorders comprising, in whole or in part, a 5 or 6 member cyclical ring carboxylic or dicarboxylic acid having at least one electronegative carbonyl subgroup or where at least one of the ring members is of an electronegatively inductive nature.

The present invention also includes compounds for the treatment of protein-mediated vertebrate diseases and disorders comprising, in whole or in part, a 5 or 6 member cyclical ring carboxylic or dicarboxylic acid having at least one electronegative carbonyl subgroup or where at least one of the ring members is of an electronegatively inductive nature, wherein the carboxylic or dicarboxylic acid that impairs the metal from joining with the protein of the vertebrate is mixed with a base, the acid ranging from 0.01% to 99.99% of the mixture and the base ranging from 0.01% to 99.99% of the mixture.

The present invention also includes compounds for the treatment of protein-mediated vertebrate diseases and disorders comprising, in whole or in part, a 5 or 6 member cyclical ring carboxylic or dicarboxylic acid having at least one electronegative carbonyl subgroup or where at least one of the ring members is of an electronegatively inductive nature, wherein the electronegatively inductive ring member is selected from the group consisting of nitrogen, oxygen, and sulfur and wherein the carboxylic or dicarboxylic acid is selected from the group consisting of picolinic acid, dinicotinic acid, dipicolinic acid, 2,4 pyrazoledicarboxylic acid, lutidinic acid, isocinchomeranic acid, quinolinic acid, and lipoic acid.

The present invention also includes compounds for the treatment of protein-mediated vertebrate diseases and disorders comprising, in whole or in part, a 5 or 6 member cyclical ring carboxylic or dicarboxylic acid having at least one electronegative carbonyl subgroup or where at least one of the ring members is of an electronegatively inductive nature, wherein the carboxylic or dicarboxylic acid that impairs the metal from joining with the protein of the vertebrate is mixed with a base, the acid ranging from 0.01% to 99.99% of the mixture and the base ranging from 0.01% to 99.99% of the mixture, and wherein the protein is a nascent or existing matrix metalloprotein and the mixture has a pH that is selected to optimize the ability of the mixture to cross the cell membrane of the vertebrate in a safe and effective manner and wherein the base is selected from the group consisting of ammonia cation, amine cation, and hydrazine cation.

The present invention also includes a method to inhibit a vertebrate from sunburning comprising administering to the vertebrate, in a safe and effective manner, ultraviolet light absorbing compounds that are capable of crossing a vertebral cell membrane.

The present invention also includes a method to inhibit a vertebrate from sunburning comprising administering to the vertebrate, in a safe and effective manner, ultraviolet light absorbing compounds that are capable of crossing a vertebral cell membrane, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature.

The present invention also includes a method to inhibit a vertebrate from sunburning comprising administering to the vertebrate, in a safe and effective manner, ultraviolet light absorbing compounds that are capable of crossing a vertebral cell membrane, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature, and wherein the carboxylic or dicarboxylic acid that inhibits the vertebrate from sunburning is mixed with a base, the acid ranging from 0.01% to 99.99% of the mixture and the base ranging from 0.01% to 99.99% of the mixture.

The present invention also includes a method to inhibit a vertebrate from sunburning comprising administering to the vertebrate, in a safe and effective manner, ultraviolet light absorbing compounds that are capable of crossing a vertebral cell membrane, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature, and wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

The present invention also includes a method to inhibit a vertebrate from sunburning comprising administering to the vertebrate, in a safe and effective manner, ultraviolet light absorbing compounds that are capable of crossing a vertebral cell membrane, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature, and wherein the carboxylic or dicarboxylic acid that inhibits the vertebrate from sunburning is mixed with a base, the acid ranging from 0.01% to 99.99% of the mixture and the base ranging from 0.01% to 99.99% of the mixture, and wherein the mixture has a pH that is selected to optimize the ability of the mixture to cross the cell membrane of the vertebrate in a safe and effective manner and wherein the base is selected from the group consisting of ammonia cation, amine cation, and hydrazine cation.

The present invention also includes a method to inhibit a vertebrate from sunburning comprising administering to the vertebrate, in a safe and effective manner, ultraviolet light absorbing compounds that are capable of crossing a vertebral cell membrane, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature, and wherein the compound is formulated for administration from the group consisting of pill, capsule, liquid, aerosol, powder, spray, mist, creme, ointment, atomized vapor, and suspension.

The present invention also includes compounds to inhibit a vertebrate from sunburning comprising, in whole or in part, a 5 or 6 member cyclical ring carboxylic or dicarboxylic acid having at least one electronegative carbonyl subgroup or where at least one of the ring members is of an electronegatively inductive nature.

The present invention also includes compounds to inhibit a vertebrate from sunburning comprising, in whole or in part, a 5 or 6 member cyclical ring carboxylic or dicarboxylic acid having at least one electronegative carbonyl subgroup or where at least one of the ring members is of an electronegatively inductive nature, wherein the carboxylic or dicarboxylic acid that inhibits the vertebrate from sunburning is mixed with a base, the acid ranging from 0.01% to 99.99% of the mixture and the base ranging from 0.01% to 99.99% of the mixture.

The present invention also includes compounds to inhibit a vertebrate from sunburning comprising, in whole or in part, a 5 or 6 member cyclical ring carboxylic or dicarboxylic acid having at least one electronegative carbonyl subgroup or where at least one of the ring members is of an electronegatively inductive nature, wherein the electronegatively inductive ring member is selected from the group consisting of nitrogen, oxygen, and sulfur, and wherein the carboxylic or dicarboxylic acid is selected from the group consisting of picolinic acid, dinicotinic acid, dipicolinic acid, 2,4 pyrazoledicarboxylic acid, lutidinic acid, isocinchomeranic acid, quinolinic acid, and lipoic acid and wherein the base is selected from the group consisting of ammonia cation, amine cation, and hydrazine cation.

It is a characteristic of the present invention that the compound, in part or in total, is a planar molecule.

It is a characteristic of the present invention that the compound, in part or in total, is aromatic.

It is a characteristic of the present invention that the compound, in part or in total, provides the metals that it associates with four electrons.

It is a characteristic of the present invention that the compound, in part or in total, covalently bonds the metal it associates with.

It is a characteristic of the present invention that the compound, in part or in total, is bound to the metal it associates with by electronegative induction.

It is a characteristic of the present invention the compound is, in part or in total, substantially both lipid and water soluble.

It is a characteristic of the present invention that the compound is, in part or in total, capable of inhibiting the formation of a matrix metalloprotein (MMP) by chelating the metal, thereby keeping the metal from associating with a protein to form an MMP.

It is a characteristic of the present invention that the compound is, in part or in total, chemically stable.

It is a characteristic of the present invention that the compound is, in part or in total, resistant to metabolic decomposition.

It is a characteristic of the present invention that the compound preferentially chelate with light-to-transition metals.

It is a characteristic of the present invention that the compound, in part or in total, sterically hinder light-to-transition metals from associating with protein to form an MMP.

It is a characteristic of the present invention that the compound is, in part or in total, capable of inhibiting the formation of a matrix metalloprotein (MMP) by ionically associating with the metal, thereby keeping the metal from associating with a protein to form an MMP.

It is a characteristic of the present invention that the compound's molecules coordinate with each other, thereby better shielding the metals they complex with from associating with a protein to form an MMP.

It is a characteristic of the present invention the compound is, in part or in total, chemically stable.

It is a characteristic of the present invention the compound is, in part or in total, resistant to metabolic decomposition.

It is a characteristic of the present invention that the compound, in part or in total, chelates to light-to-transition metals in preference to heavy metals.

It is a characteristic of the present invention that the compound, in part or in total, inhibits the function of protein kinases, phosphatases, phospholipases, and extracellular lipases.

It is a characteristic of the present invention that the compounds used, in part or in total, inhibit the binding of free metals to protein receptor sites, protein, and phospholipases.

It is a characteristic of the present invention that the compounds used, in part or in total, increase both internally and externally the cell of a vertebrate's resistance to ultraviolet radiation.

It is a characteristic of the present invention that the compounds when used, in part or in total, in topical applications, are resistant to being washed off.

### DETAILED DESCRIPTION OF THE INVENTION

The description of the present invention is organized as follows: a general description is described: then alternative embodiments are described.

The invention involves methods and compounds for treating vertebral diseases that are caused by the vertebrate's own protein functioning against it. Specifically, the invention involves inhibiting the function of metal-dependent vertebrate protein by inhibiting the metal from joining to these proteins and/or by impairing the function of these metals after the metal has already joined to the protein. The compounds are substantially both water and lipid soluble. Dipicolinic acid, for example is soluble in both water and glycerin. The compounds thus have the solubility needed to penetrate the cell membrane of a vertebrate, where vertebral proteins such as MMPs reside, and interact/bind/sequester the free metals needed by proteins, such as MMPs, to function.

Because this solubility allows the compounds meaningful access to intracellular free metals, reduction of, for example, cellular inflammation via interference with MMP formation is subtly simpler than irreversibly binding the metal using strong chelators because such proteins are transient. Thus, for example, an anti-proteinase effect can be accomplished merely by interfering with the metal joining with the potential MMP long enough for the transient protein portion to be recycled by the cell.

The present invention also involves methods and compounds for inhibiting/preventing sunburn in a vertebrate due to their chemical structural similarity to the active ingredients found in sunscreens. Specifically, the invention involves inhibiting ultraviolet damage to a vertebrate by administering ultraviolet absorbing compounds that, due to their ability to penetrate a cell membrane because they are substantially both water and lipid soluble and have a chemical predilection for the calcium found in the interior of a vertebrate's cell, provide the vertebrate with UV protection from the interior of the vertebrate's cells. Further, dipicolinic acid, one of the compounds claimed in the present invention, is, for example, the main reason for an endospore's high resistance to UV radiation. Thus, though capable of providing the vertebrate with UV protection from the exterior of the vertebrate's cells, as do presently known sunscreens, the present invention also contemplates being administered as a pill, as well as the convention application modes of capsule, liquid, aerosol, powder, spray, mist, creme, ointment, atomized vapor, and suspension.

The compounds included in the invention are five and six-membered cyclical ring carboxylic acids, the ring having at least one high electronegativity inductive molecule including, but is not limited to, nitrogen, oxygen and sulfur, or has at least one carbonyl subgroup attached to the ring. Thus, whether as a subgroup or as an actual ring member, all of the present invention's compounds have an electronegativity inductive nature.

The pH of the compounds used in the present invention may be adjusted by bases that include, but are not limited to, ammonia, an amine, or hydrazine. The percentage of base in this mixture can vary to between 0.01% to 99.99%. The percentage of acid in this mixture can vary to between 0.01% to 99.99%.

The compounds can include, but are not limited to, in whole or in part, picolinic acid, dipicolinic acid, dinicotinic acid, 2,4 pyrazoledicarboxylic acid, lutidinic acid, isocinchomeranic acid, quinolinic acid, and lipoic acid.

Protein-mediated diseases/disorders means biochemical events controlled and/or produced by vertebral protein which, when wrongly or overly performed, leads the vertebrate into a diseased or disordered state. Such protein-mediated diseases and disorders include, but are not limited to, rheumatoid arthritis, osteoarthritis, aneurysm, atherosclerosis, pulmonary emphysema, acne vulgaris, acne rosacea, lupus erythematosis, Alzheimer's disease, sterile corneal ulcer, iritis, blepharitis, aneurysm formation, tumor metastasis, tumor angiogenesis and the over-production of C-reactive protein.

Complex means to have a first molecule, in conjunction with other first molecules, chemically coordinate, in a crystallization-like manner, with a second dissimilar molecule or molecules, in such a way as to inhibit the more centrally located second molecules from coming into contact with a third chemical entity; thus, the coordination of the first molecules, working together, synergizes their inhibitory effect of keeping the second dissimilar molecules from coupling with the third chemical entity.

Safe and effective means administering to a vertebrate an amount of the present invention's compounds that reduces, prevents or eliminates the vertebrate's protein-mediated disease/disorder without producing significant toxicity to the vertebrate.

The compound's formulations can include, but are not limited to, pill, capsule liquid, aerosol, powder, spray, mist, creme, ointment, atomized vapor, or suspension.

In a preferred embodiment, the compound is dipicolinic acid mixed with a hydrazine base and is administered to the vertebrate in a safe and effective manner, the pH of the mixture substantially matching the pH of a vertebrate.

In another preferred embodiment, the electronegatively inductive component of the molecule is composed of at least one carbonyl subgroup attached to a 6-member aromatic ring dicarboxylic acid that is mixed with a ammonia cation and is administered to the vertebrate in a safe and effective manner, the pH of the mixture substantially matching the pH of a vertebrate.

Although this invention has been described with a certain degree of particularity, it is understood that numerous changes might still occur to a person of ordinary skill in the art without departing from the spirit and scope of the invention.

I claim:

1. A method for treating protein-mediated vertebrate diseases and disorders, comprising administering a chemical compound capable of electronegatively interacting with a metallic cation in a manner and amount sufficient to substantially impair, in a safe and effective manner, the metallic cation from effectively joining with the protein, thereby inhibiting the function of the protein.

2. The method of claim 1, wherein the compound that impairs the metal from joining with the protein is, in whole or in part, a 5 or 6 member cyclical ring carboxylic or dicarboxylic acid having at least one electronegative carbonyl subgroup or where at least one of the ring members is of an electronegatively inductive nature.

3. The method of claim 2, wherein the carboxylic or dicarboxylic acid that impairs the metal from joining with the protein of the vertebrate is mixed with a base, the acid ranging from 0.01% to 99.99% of the mixture and the base ranging from 0.01% to 99.99% of the mixture.

4. The method of claim 2, wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

5. The method of claim 3, wherein the protein is a nascent or existing matrix metalloprotein and the mixture has a pH that is selected to optimize the ability of the mixture to cross the cell membrane of the vertebrate in a safe and effective manner and wherein the base is selected from the group consisting of ammonia cation, amine cation, and hydrazine cation.

6. A method for treating protein-mediated vertebrate diseases and disorders, comprising administering a chemical compound capable of chelating with a metallic cation in a manner and amount sufficient to substantially impair in a safe and effective manner the metallic cation from effectively joining with the protein, thereby inhibiting the function of the protein.

7. The method of claim 6, wherein the compound that chelates the metal from interacting with the protein is, in whole or in part, a 5 or 6 member cyclical ring carboxylic or dicarboxylic acid having at least one electronegative carbonyl subgroup or where at least one of the ring members is of an electronegatively inductive nature.

8. The method of claim 7, wherein the carboxylic or dicarboxylic acid that chelates the metal from interacting with the protein of the vertebrate is mixed with a base, the acid ranging from 0.01% to 99.99% of the mixture and the base ranging from 0.01% to 99.99% of the mixture.

9. The method of claim 7, wherein the electronegatively inductive ring member is selected from the group consisting of nitrogen, oxygen, and sulfur and wherein the carboxylic or dicarboxylic acid is selected from the group consisting of picolinic acid, dipicolinic acid, 2,4 pyrazoledicarboxylic acid, lutidinic acid, isocinchomeranic acid, quinolinic acid, and lipoic acid.

10. The method of claim 8, wherein the protein is a nascent or existing matrix metalloprotein and the mixture has a pH that is selected to optimize the ability of the mixture to cross the cell membrane of the vertebrate in a safe and effective manner and wherein the base is selected from the group consisting of ammonia cation, amine cation, and hydrazine cation.

11. A method for treating protein-mediated vertebrate diseases and disorders, comprising administering a chemical compound capable of sequestering a metallic cation within a complex in a manner and amount sufficient to substantially impair in a safe and effective manner the metallic cation from effectively joining with the protein, thereby inhibiting the function of the protein.

12. The method of claim 11, wherein the compound that sequesters the metal from interacting with the protein is, in whole or in part, a 5 or 6 member cyclical ring carboxylic or dicarboxylic acid having at least one electronegative carbonyl subgroup or where at least one of the ring members is of an electronegatively inductive nature.

13. The method of claim 12, wherein the carboxylic or dicarboxylic acid that sequesters the metal from proximity with the protein of the vertebrate is mixed with a base, the acid ranging from 0.01% to 99.99% of the mixture and the base ranging from 0.01% to 99.99% of the mixture.

14. The method of claim 12, wherein the electronegatively inductive ring member is selected from the group consisting of nitrogen, oxygen, and sulfur and wherein the carboxylic or dicarboxylic acid is selected from the group consisting of picolinic acid, dipicolinic acid, 2,4 pyrazoledicarboxylic acid, lutidinic acid, isocinchomeranic acid, quinolinic acid, and lipoic acid.

15. The method of claim 13, wherein the protein is a nascent or existing matrix metalloprotein and the mixture has a pH that is selected to optimize the ability of the mixture to cross the cell membrane of the vertebrate in a safe and effective manner and wherein the base is selected from the group consisting of ammonia cation, amine cation, and hydrazine cation.

16. The method of claim 2, wherein the compound is formulated for administration from the group consisting of pill, capsule, liquid, aerosol, powder, spray, mist, creme, ointment, atomized vapor, and suspension.

17. The method of claim 7, wherein the compound is formulated for administration from the group consisting of pill, capsule, liquid, aerosol, powder, spray, mist, creme, ointment, atomized vapor, and suspension.

18. The method of claim 12, wherein the compound is formulated for administration from the group consisting of pill, capsule, liquid, aerosol, powder, spray, mist, creme, ointment, atomized vapor, and suspension.

19. Compounds for the treatment of protein-mediated vertebrate diseases and disorders comprising, in whole or in part, a 5 or 6 member cyclical ring carboxylic or dicarboxylic acid having at least one electronegative carbonyl subgroup or where at least one of the ring members is of an electronegatively inductive nature.

20. The compound of claim 19, wherein the carboxylic or dicarboxylic acid that impairs the metal from joining with the protein of the vertebrate is mixed with a base, the acid ranging from 0.01% to 99.01% of the mixture and the base ranging from 0.01% to 99.99% of the mixture.

21. The compound of claim 19 wherein the electronegatively inductive ring member is selected from the group consisting of nitrogen, oxygen, and sulfur and wherein the carboxylic or dicarboxylic acid is selected from the group consisting of picolinic acid, dinicotinic acid, dipicolinic acid, 2,4 pyrazoledicarboxylic acid, lutidinic acid, isocinchomeranic acid, quinolinic acid, and lipoic acid.

22. The compound of claim 20 wherein the protein is a nascent or existing matrix metalloprotein and the mixture has a pH that is selected to optimize the ability of the mixture to cross the cell membrane of the vertebrate in a safe and effective manner and wherein the base is selected from the group consisting of ammonia cation, amine cation, and hydrazine cation.

23. A method to inhibit a vertebrate from sunburning comprising administering to the vertebrate, in a safe and effective manner, ultraviolet light absorbing compounds that are capable of crossing a vertebral cell membrane.

24. The method of claim 23, wherein the compound that inhibits the vertebrate from sunburning is, in whole or in part, a 5 or 6 member cyclical ring carboxylic or dicarboxylic acid having at least one electronegative carbonyl subgroup or where at least one of the ring members is of an electronegatively inductive nature.

25. The method of claim 24, wherein the carboxylic or dicarboxylic acid that inhibits the vertebrate from sunburning is mixed with a base, the acid ranging from 0.01% to 99.01% of the mixture and the base ranging from 0.01% to 99.99% of the mixture.

26. The method of claim 24, wherein the electronegatively inductive ring member is selected from the group consisting of nitrogen, oxygen, and sulfur and wherein the carboxylic or dicarboxylic acid is selected from the group consisting of picolinic acid, dipicolinic acid, dinicotinic acid, 2,4 pyrazoledicarboxylic acid, lutidinic acid, isocinchomeranic acid, quinolinic acid, and lipoic acid.

27. The method of claim 25, wherein the mixture has a pH that is selected to optimize the ability of the mixture to cross the cell membrane of the vertebrate in a safe and effective manner and wherein the base is selected from the group consisting of ammonia cation, amine cation, and hydrazine cation.

28. The method of claim 24, wherein the compound is formulated for administration from the group consisting of pill, capsule, liquid, aerosol, powder, spray, mist, creme, ointment, atomized vapor, and suspension.

29. Compounds to inhibit a vertebrate from sunburning comprising, in whole or in part, a 5 or 6 member cyclical ring carboxylic or dicarboxylic acid having at least one electronegative carbonyl subgroup or where at least one of the ring members is of an electronegatively inductive nature.

30. The compound of claim 29, wherein the carboxylic or dicarboxylic acid that inhibits the vertebrate from sunburning is mixed with a base, the acid ranging from 0.01% to 99.99% of the mixture and the base ranging from 0.01% to 99.99% of the mixture.

31. The compound of claim 29 wherein the electronegatively inductive ring member is selected from the group consisting of nitrogen, oxygen, and sulfur and wherein the carboxylic or dicarboxylic acid is selected from the group consisting of picolinic acid, dinicotinic acid, dipicolinic acid, 2,4 pyrazoledicarboxylic acid, lutidinic acid, isocinchomeranic acid, quinolinic acid, and lipoic acid and wherein the base is selected from the group consisting of ammonia cation, amine cation, and hydrazine cation.
